(19) United States
(12) Patent Application Publication
(10) Pub. No.: US 2025/0000712 A1
(43) Pub. Date: Jan. 2, 2025

(54) METHOD AND APPARATUS FOR PRODUCING A BUNDLE PACK FOR HYGIENE PRODUCTS, AND CORRESPONDING BUNDLE PACK

(71) Applicant: Focke & Co. (GmbH & Co. KG), Verden (DE)

(72) Inventor: Josef Schulte, Aschendorf (DE)

(73) Assignee: Focke & Co. (GmbH & Co. KG), Verden (DE)

(21) Appl. No.: 18/700,154

(22) PCT Filed: Sep. 30, 2022
(86) PCT No.: PCT/EP2022/077339

§ 371 Date: Apr. 10, 2024

(30) Foreign Application Priority Data
Oct. 15, 2021 (DE) 10 2021 126 762.5

(51) Int. Cl. A61F 13/15

(52) U.S. Cl. CPC A61F 13/15577

(57) ABSTRACT

A method and apparatus for producing a cuboidal bundle pack, and the bundle pack produced thereby, for compressible hygiene products, wherein a group of hygiene products is inserted into a sleeve, open on two sides, consisting of packaging material, and the sleeve is then sealed for the purpose of forming a wrapping, preferentially closed on all sides, by unwinding a continuous web of material, preferentially paper-based, for the sleeve is unwound from a bobbin, applying lines of weakening to the unwound continuous web of material, separating a portion for a sleeve from the continuous web of material, the portion is righted and the preferentially compressed group of hygiene products is inserted into the righted portion via an open side of the sleeve, and sealing opposing sides of the sleeve by means of an envelope fold.

### CROSS REFERENCE TO RELATED APPLICATIONS

This application is the US national phase of and claims the benefit of and priority on International Application No. PCT/EP2022/077339 having an international filing date of 30 Sep. 2022, which claims priority on and the benefit of German Patent Application No. 10 2021 126 762.5 having a filing date of 15 Oct. 2021.

### BACKGROUND OF THE INVENTION

### Technical Field

The invention relates to a method for producing a cuboidal bundle pack for compressible hygiene products, wherein a group of hygiene products is inserted into a sleeve, open on two sides, consisting of packaging material, and the sleeve is then sealed for the purpose of forming a wrapping, preferentially closed on all sides.

The invention further relates to a corresponding apparatus for producing a cuboidal bundle pack for compressible hygiene products, wherein a group of hygiene products is capable of being inserted into a sleeve, open on two sides, consisting of packaging material, and the sleeve is then capable of being sealed for the purpose of forming a wrapping, preferentially closed on all sides, and also to such a bundle pack.

Within the scope of this application, “hygiene products” is understood to mean, in particular, products from the fields of feminine hygiene, baby care and adult care, such as, for instance, sanitary napkins, panty liners, baby diapers and incontinence pads.

### Prior Art

From the prior art it is known to package groups of hygiene products in a wrapping consisting of polyethylene. In this process, the group of products is inserted into a bag that is open on one side, and the bag is subsequently sealed in a sealing station, for instance by formation of a sealing seam. Protruding ends of the seam can subsequently be shortened.

Polyethylene bags of such a type are not optimal as regards the increasingly important topic of sustainability. Known production methods have the disadvantage, in particular, that a lot of energy is needed for the sealing method. In addition, waste arises when the seam is shortened. The bags that are employed have to be produced in advance in a separate method, and have to be supplied to the packaging machine in elaborate manner in the form of stacks, or individually.

### BRIEF SUMMARY OF THE INVENTION

Proceeding from here, the object underlying the invention is to refine methods, apparatuses and products of the type stated in the introduction, in particular with regard to production that is as sparing of resources as possible, and/or with regard to elimination of the aforementioned problems.

A method for achieving this object exhibits the following method steps:
a continuous web of material, preferentially paper-based, for the sleeve is unwound from a bobbin,
lines of weakening are applied to the unwound, continuous web of material,
a portion for a sleeve is separated from the continuous web of material,
the portion is righted, and the preferentially compressed group of hygiene products is inserted into the righted portion via an open side of the sleeve,
opposing end faces of the sleeve are each sealed by means of an envelope fold.

A further special feature may consist in the fact that the envelope folds are held together by adhesive and/or by an adhesive bond, in particular by a covering label.

There may preferentially be provision that a web of material having a tubular cross-section is employed, from which tubular portions for the sleeve are separated.

The special feature accordingly consists in the fact that the material for the sleeve is supplied as a continuous web of material. The web of material accordingly takes the form of a flexible tube and is consequently double-layered. In this way, the width of the web of material is halved in comparison with a single-layer web of material, and production is simplified, because the formation of the flexible tube does not occur only during the packaging process.

There is preferentially provision that the tubular portion is righted to form an open flexible tube or, to be more exact, the sleeve.

There is preferentially provision, furthermore, that the group of hygiene products is inserted into the sleeve in a compressed state and then expands within the sleeve.

In a first variant of a development, there may be provision that folding flaps of the sleeve for the envelope fold are firstly pre-folded with fixed and/or movable folding elements, are then at least partially provided with adhesive, and are finally folded to form an envelope, so that the two ends of the sleeve have each been sealed completely by the folding flaps of the envelope fold which have been connected to one another with adhesive.

Alternatively, there may be provision that folding flaps of the sleeve for the envelope fold are folded in the manner of an envelope with fixed and/or movable folding elements, whereby the folding flaps only partially overlap one another and leave a region free between the folding flaps, and that the folded folding flaps are then at least partially provided with adhesive, and a covering label for at least partial covering of the envelope fold and of the region left free is stuck onto an end face of the bundle pack.

It will be understood that the covering label may also be employed when the folding flaps of the envelope overlap one another in such a way that no free region arises.

Moreover, there may be provision that the web of material is imprinted on one side or on both sides during the process. Decorative and/or informative imprints, in particular, are conceivable.

Moreover, there may be provision that a perforation by way of opening-aid for the bundle pack is applied to the web of material or to the portions separated therefrom. In this way, the bundle pack for the consumer can be opened easily.

As an alternative to a prefabricated tubular web of material, an unfolded web of material can be unwound from a bobbin and folded during the process to form a tubular web of material from which individual sleeves are separated.

An apparatus for achieving the object stated in the introduction exhibits the features of:
a transport system for a continuous web of material, preferentially paper-based, wound on a bobbin,
one or more stations for applying lines of weakening to the unwound, continuous web of material,
a separating station for isolating portions for a sleeve from the continuous web of material,
a righting station which has been set up to right a portion and to insert a preferentially compressed group of hygiene products into the righted portion via an open side of the sleeve,
a closure station which has been set up to seal opposing end faces of the sleeve by means of an envelope fold in each instance.

Moreover, there may be provision that a folding station for the folding of folding flaps of the sleeve has been provided, as well as a gluing station for applying adhesive onto the folding flaps.

A bundle pack according to the invention is a bundle pack for a group of compressible hygiene products, wherein the group of hygiene products exhibits a cuboidal shape and is surrounded by a sleeve consisting of packaging material, preferentially paper-based, the open sides of which have been at least partially sealed by means of envelope folds, produced, in particular, by a method as taught herein.

There may preferentially be provision that the sleeve for the bundle pack exhibits an adhesively bonded seam in the longitudinal direction of a web of material from which the sleeve has been separated.

There may preferentially be provision that the sleeve for the bundle pack exhibits lines of weakening, in order to make possible a preferentially sharp-edged fold around the cuboidal group of hygiene products.

There may preferentially be provision that the end faces of the sleeve with the envelope folds have been sealed in each instance by a covering label which has been connected by means of adhesive to the folding flaps folded in the manner of an envelope.

There may preferentially be provision that the end faces of the sleeve have been sealed by means of envelope folds overlapping one another which have been at least partially connected to one another by means of adhesive.

There may preferentially be provision that cold-glue-based adhesive is employed.

There may preferentially be provision that a self-adhesive label, preferentially paper-based, is employed instead of a covering label fastened with adhesive.

### BRIEF DESCRIPTION OF THE DRAWINGS

Preferred embodiments of the invention will be described in the following with reference to the drawing:

FIG. 1 shows a first embodiment of a bundle pack in schematic spatial representation;

FIG. 2 shows a second embodiment of a bundle pack in a representation corresponding to FIG. 1;

FIG. 3 shows a first embodiment of an apparatus for producing a bundle pack according to FIG. 1, in schematic spatial representation;

FIG. 4 shows a second embodiment of an apparatus for producing a bundle pack according to FIG. 2, in a representation corresponding to FIG. 3;

FIG. 5 shows an embodiment of an apparatus for producing the requisite sleeve-like web of material, in schematic spatial representation;

FIG. 6 shows a first variant of the web of material in a section along line of intersection VI-VI in FIG. 5; and

FIG. 7 shows a second variant of the web of material, in a representation analogous to FIG. 6.

### DETAILED DESCRIPTION OF PREFERRED EMBODIMENTS

The invention will be described below on the basis of two variants of bundle packs 10 for a group 11 of hygiene products 12. Sanitary napkins, panty liners, baby diapers, incontinence pads or incontinence diapers, in particular, come into consideration as hygiene products 12.

Within the group 11 the hygiene products 12 have been arranged in a regular formation, so that a cuboidal group 11 has been formed. The group 11 is directly surrounded by a wrapping 13 which is formed from a sleeve 14 consisting of packaging material. The sleeve 14 possesses two initially open, opposing end faces 15 through which the hygiene products 12 can be supplied. After this, the end faces 15 are sealed, namely in each instance by means of an envelope fold of folding flaps 16, 17, 18, 19 of the material of the sleeve 14, so that a wrapping 14, extending all around, of the group 11 of hygiene products 12 arises.

Paper or a paper-based material—that is to say, in particular, a coated paper—comes into consideration as packaging material.

The packaging material for the sleeve 14 is made available as a continuous web of material 20 from which individual portions of the sleeve 14 are separated. The web of material 20 has been formed as an (endless) sleeve 14 having a tubular cross-section. Correspondingly, the web of material 20 exhibits a flexible-tube seam 21 extending in the longitudinal direction, which is also to be found on the sleeves 14 separated therefrom. The sleeve-like web of material 20 can be made available on a bobbin (not shown), so that in the course of the production of the bundle packs 10 the production of the longitudinally extending flexible-tube seam 21 on site becomes unnecessary.

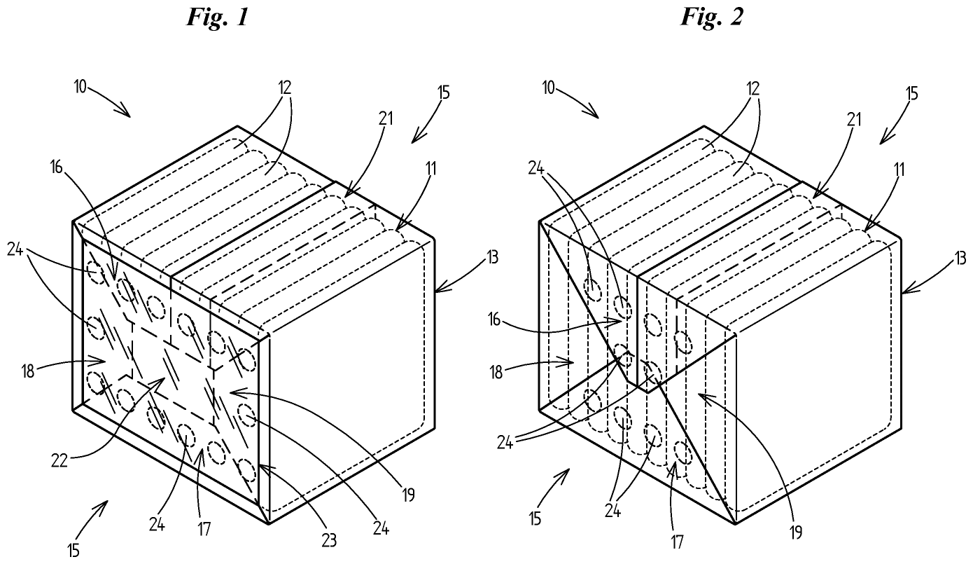

One difference between the two alternatives represented in FIGS. 1 and 2 relates to the closing of the wrapping 13 in the region of the end faces 15.

In the variant according to FIG. 1, a sleeve 14 is employed having shorter folding flaps 16, 17, 18, 19 in the region of the end faces 15 than in the variant according to FIG. 2. Whereas in FIG. 2 the folding flaps 16, 17, 18, 19 are so long that the folded folding flaps 16, 17, 18, 19 placed on top of one another overlap one another in such a way that the wrapping 13 has been closed completely in the region of the end faces 15, in the variant shown in FIG. 1 a central region 22 remains, in which the wrapping 13 has not been closed. Instead of this, a covering label 23 has been provided which covers over the region 22 and a large proportion of the area of the folding flaps 16, 17, 18, 19.

For the purpose of connecting the covering label 23 to the wrapping 13, adhesive areas 24—for instance, spots of cold-glue-based glue which are applied onto the folded folding flaps 16, 17, 18, 19—are employed which are applied encircling the region 22. Adhesive areas 24 are employed also in the variant according to FIG. 2, which, however, serve to connect the folding flaps 16, 17, 18, 19 to one another. Of course, a covering label 23 may also be employed in the variant according to FIG. 2.

Instead of a covering label 23 stuck on with spots of glue, a self-adhesive label, preferentially paper-based, for instance with a coating of adhesive, may also be employed (in both variants).

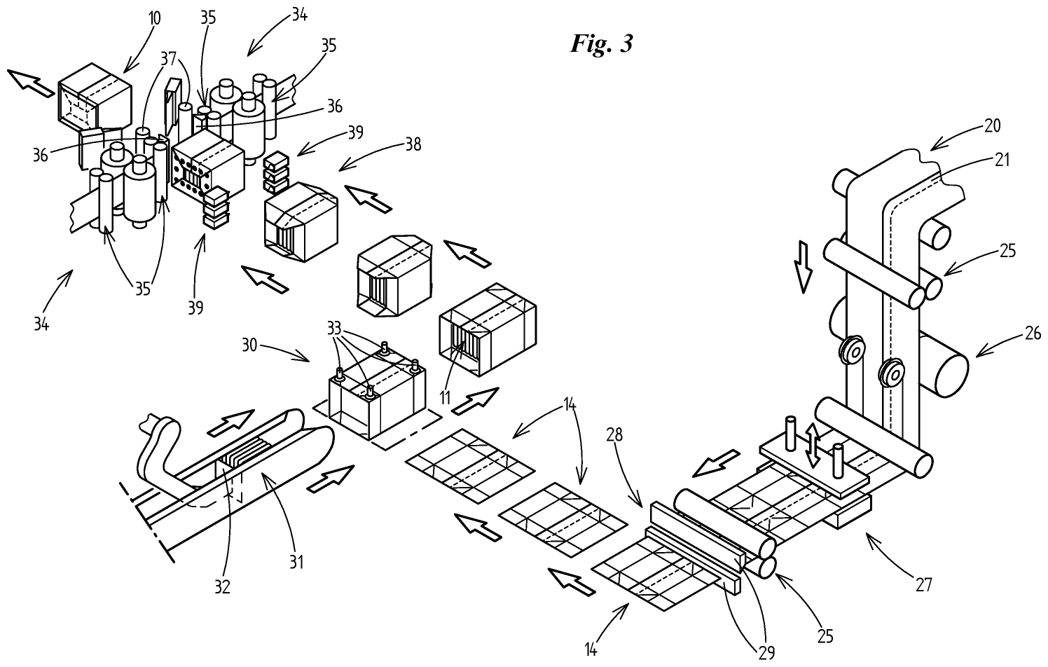

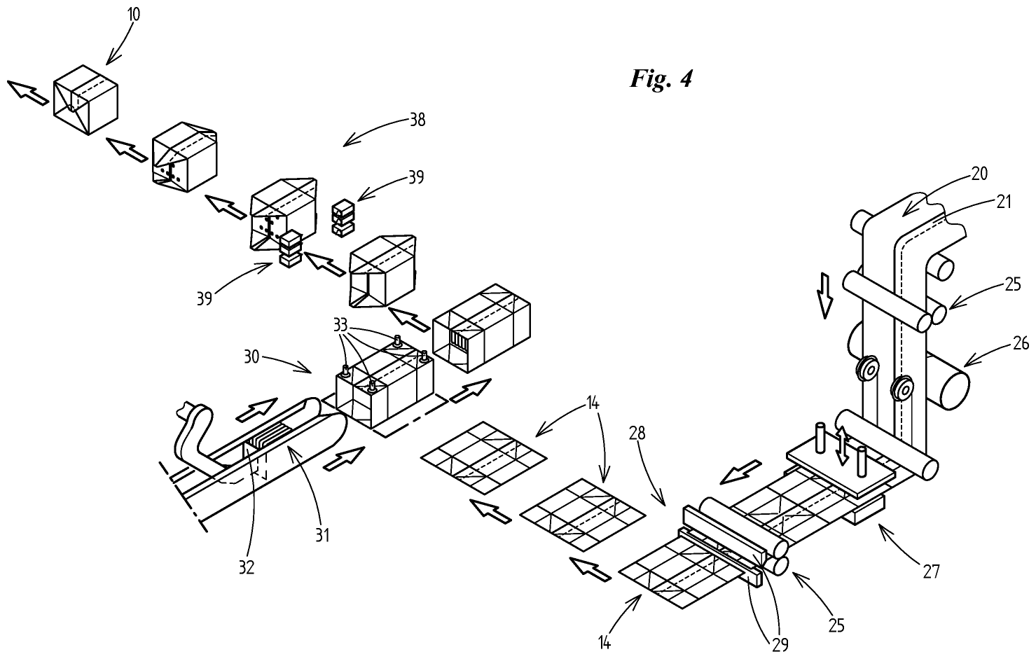

FIGS. 3 and 4 show, respectively, apparatuses and methods for producing the bundle packs 10 according to FIGS. 1 and 2, respectively. A web of material 20 (for example, consisting of kraft paper) which is tubular in cross-section is firstly unwound intermittently from a wound roll/bobbin by means of draw-forward rollers 25 and is supplied firstly to a first web-treatment station 26, where the web of material 20 is provided with longitudinal grooves, and then to a second web-treatment station 27, which applies groove lines extending at right angles to the running direction of the web by embossing the web of material 20. In this way, the top folds and bottom folds of the end fold of the sleeves 14 are prepared.

The tension of the web is regulated by dancer rollers of a pendulum (not shown). After the treatment of the web, the web of material 20 is supplied to a separating station 28 where a flexible-tube portion for a sleeve 14 is generated, for instance by separating a portion of the web of material 20 by means of separating blades 29.

The separated flexible-tube portions are then pushed away transversely and transported away further to a righting station 30 (for example, by means of suction straps). At the righting station 30, each flexible-tube portion—that is to say, each sleeve 14—is righted with the aid of suction devices 33 known from the prior art, or is spread out and held by auxiliary means known from the prior art, so that compressed hygiene products can be inserted into the sleeve 14 with the aid of a shuttle 31 and a slide 32, likewise known from the prior art.

The hygiene products 12 are supplied in groups to a compressing station known from the prior art. In this station, the groups 11 are then pressed together and immediately afterwards are pushed off (not shown) into the shuttle 31 by means of the slide 32. The shuttle 31 and the slide 32 together push the compressed group 11 into the sleeve 14. When the group 11 reaches the central region of the sleeve 14, the shuttle 31 is drawn back again in the direction of the compressing station, while the slide 32 pushes the group 11 out of the channel of the shuttle 31. In this process, the compressed group 11 expands and fits snugly against the walls of the sleeve 14 from the inside. Immediately afterwards, the slide 32 pushes the group 11 with the sleeve 14 further to the next folding stretch or folding path where the envelope fold of the folding flaps 16, 17, 18, 19 in the region of the end faces 15 is accomplished.

The envelope fold of the folding flaps 16, 17, 18, 19 is realized with the aid of the fixed and movable elements, which are not shown since they are known from the prior art. First of all, the lateral folding flaps 18, 19 are folded into the plane of the end faces 15 by means of the stated elements. Subsequently the lower and upper projecting folding flaps 16, 17 are flipped over in succession. This is done in a closure station 38.

In the course of the second method sequence shown in FIG. 4, prior to the final folding of the outer—that is to say, lower and upper—folding flaps 16, 17 the inner folding flaps 18, 19 are provided with the adhesive areas 24 which are intended to hold the folding flaps together after the final folding (for example, after setting of the adhesive in a drying stretch which is not shown).

In the course of the method sequence shown in FIG. 3 (suitable for the bundle according to FIG. 1), the folding flaps 16, 17, 18, 19 have been made shorter than in the second variant of the bundle (FIG. 2). This results in an opening in the center of the end faces 15, which is covered by the covering label 23. The gluing of the folding flaps 16, 17, 18, 19 for the purpose of fastening the covering label 23 occurs subsequent to the final folding of the folding flaps 16, 17, 18, 19 in a gluing station 39. In two stations 34, the covering labels 23 are cut from a wound roll (not shown), and the covering label 23 is applied to the end faces 15 of the bundle pack 10 by means of draw-forward rollers 35, a guide rail 36 and a pressure roller 37 as the bundle pack 10 moves past. The setting of the adhesive may occur in a subsequent drying stretch (not shown).

Alternatively, the application of the adhesive to the covering label 23 may also occur before these labels are placed on the end faces 15 of the bundle pack 10 and pressed on.

As a substitute for the cold adhesive, after the unrolling of the web of material 20 and prior to the isolating of the sleeves 14 two further stations may be provided which can be arranged on both sides of the web of material 20 and which apply a reactivatable trail of glue or spots of glue at the corresponding end-fold points on the web of material 20. After the sealing of the end fold of the bundle, or after the placing of the covering label in position, further contact elements can be provided on both sides of the transport path, which by influence of heat reactivate the reactivatable glue for the purpose of connecting the folding flaps 16, 17, 18, 19 and the covering label 23.

Alternatively, a contactless reactivation of the applied glue may also be provided, specifically before the outer flaps are sealed or before the covering labels are placed or applied onto the end faces of the bundle.

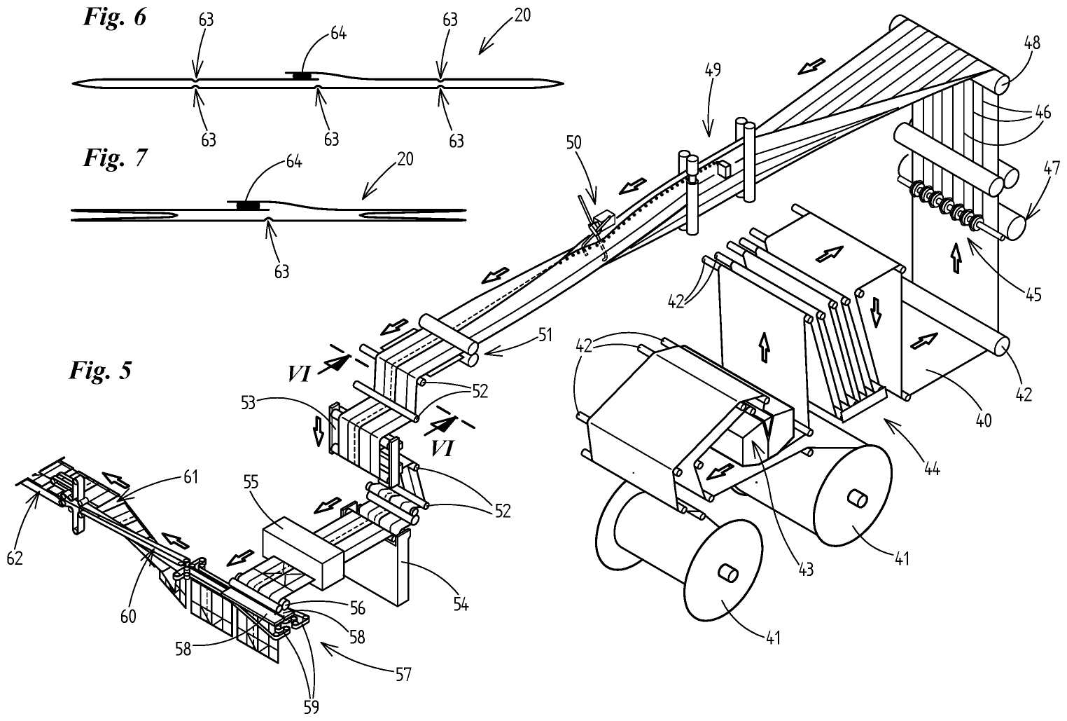

FIGS. 5 to 7 are concerned with the subject of the production of a sleeve-like web of material 20. Instead of drawing off the web of material 20 in prefabricated form from a bobbin, as in FIGS. 3 and 4, the web of material 20 can also be produced on site from a flat, unfolded web of material 40. For instance, an appropriate apparatus may be arranged directly ahead of the apparatus according to FIGS. 3 and 4.

According to FIG. 5, the web of material 40 is made available on two bobbins 41 and is supplied to a splicing station 43 via deflection rollers 42. From there, it goes into a reciprocating store 44 via further deflection rollers. Immediately following the reciprocating store 44, longitudinal grooves 46 by way of weakening grooves are applied to the web of material 40 in an upright conveying section with a longitudinal-groove unit 45, preferentially during the continuous transport of the web of material 40. Draw-forward rollers 47 for transporting the web of material 40 provided with the longitudinal grooves 46 are located immediately following the longitudinal-groove unit 45.

Via a further deflection roller 48, the web of material 40, provided with the longitudinal grooves 46, arrives at a horizontal conveying section where adhesive is applied in a glue-application station 49. Previously, the web of material 40 is firstly folded up (symmetrically or asymmetrically) along a crease-line. At the glue-application station 49 a trace of glue (hot glue or pressure-sensitive adhesive—PSA) is applied to the edge region of the web of material 40. Alternatively, a trace of glue having different adhesive components can also be applied to each of the two edge regions which then enter into a firm bond when the edge regions are placed on top of one another. The web of material 40 is drawn further forward to a folding station 50 where the flexible-tube shape of the web (flat flexible-tube shape according to FIG. 6 or lateral-fold shape according to FIG. 7) is generated along the crease-lines. Subsequently the edges of the web of material 40 are conducted over one another, in order to produce an overlap and consequently a flexible tube. At the pressing station 51 the longitudinal seam of the flexible tube is definitively sealed, so that a tubular or sleeve-like web of material 20 has been formed.

Immediately after this, the tubular or sleeve-like web of material 20 is passed over further deflection rollers 52, in which connection a web-edge control 53, a loop box 54 and an embossing unit 55 have also been provided along the conveying route.

Finally, the web of material 20 arrives via draw-forward rollers 56 at a clamping and cutting station 57 in which the blanks or sleeves 14 are separated from the web of material 20. Clamping strips 58 serve there to clamp the sleeves 14 from both sides in the course of separating. The isolated sleeves 14 are transported via clamping belts 59 in the direction of twisting belts 60 which turn the sleeves 14 together with a supporting guide 61 out of an upright position into a horizontal orientation. The sleeves 14 lying flat are then transported away via a suction transport belt 62.

FIG. 6 shows the web of material 20 as a simple flexible tube, whereas FIG. 7 shows the web of material 20 as a side-pleated flexible tube. Furthermore, the generated grooves 63 and also the glue 64 are shown.

### LIST OF REFERENCE SYMBOLS

10 bundle pack
11 group
12 hygiene product
13 wrapping
14 sleeve
15 end face
16 folding flap (top)
17 folding flap (bottom)
18 folding flap (left)
19 folding flap (right)
20 web of material
21 flexible-tube seam
22 region
23 covering label
24 adhesive areas
25 draw-forward rollers
26 web-treatment station
27 web-treatment station
28 separating station
29 separating blade
30 righting station
31 shuttle
32 slide
33 suction device
34 station
35 draw-forward roller
36 guide rail
37 pressure roller
38 closure station
39 gluing station
40 web of material
41 bobbin
42 deflection roller
43 splicing station
44 reciprocating store
45 longitudinal-groove unit
46 longitudinal groove
47 draw-forward roller
48 deflection roller
49 glue-application station
50 folding station
51 pressing station
52 deflection roller
53 web-edge control
54 loop box
55 embossing unit
56 draw-forward rollers
57 clamping and cutting station
58 clamping strips
59 clamping belts
60 twisting belt
61 supporting guide
62 suction transport belt
63 groove
64 glue

1. A method for producing a cuboidal bundle pack (10) for compressible hygiene products (12), wherein a group (11) of hygiene products (12) is inserted into a sleeve (14), open on two sides, consisting of packaging material, and the sleeve (14) is then sealed for the purpose of forming a wrapping (13), preferentially closed on all sides, comprising the steps of:
unwinding a continuous web of material (20), preferentially paper-based, for the sleeve (14) from a bobbin;
applying lines of weakening to the unwound, continuous web of material (20);
separating a portion for a sleeve (14) from the continuous web of material (20);
righting and inserting the portion and the preferentially compressed group (11) of hygiene products (12) into the righted portion via an open side of the sleeve (14); and
sealing each of opposing end faces (15) of the sleeve (14) by means of an envelope fold.

2. The method as claimed in claim 1, wherein the envelope folds are held together by adhesive and/or by an adhesive bond, in particular by a covering label (23).

3. The method as claimed in claim 1, wherein a web of material (20) having a tubular cross-section is employed, from which tubular portions for the sleeve (14) are separated.

4. The method as claimed in claim 3, wherein the tubular portion is righted to form an open flexible tube or, to be more exact, the sleeve (14).

5. The method as claimed in claim 1, wherein the group (11) of hygiene products (12) is inserted into the sleeve (14) in a compressed state and then expands in the sleeve (14).

6. The method as claimed in claim 1, wherein folding flaps (16, 17, 18, 19) of the sleeve (14) for the envelope fold are firstly pre-folded with fixed and/or movable folding elements, are then at least partially provided with adhesive, and are finally folded to form an envelope, so that the two ends of the sleeve (14) have each been sealed completely by the folding flaps (16, 17, 18, 19), connected to one another with adhesive, of the envelope fold.

7. The method as claimed in claim 6, wherein the folding flaps (16 . . . 19) of the sleeve (14) for the envelope fold are folded in the manner of an envelope with fixed and/or movable folding elements, whereby the folding flaps (16, 17, 18, 19) only partially overlap one another and leave a region free between the folding flaps (16, 17, 18, 19), and in that the folded folding flaps (16, 17, 18, 19) are then at least partially provided with adhesive, and a covering label (23) for at least partial covering of the envelope fold and of the region left free is stuck onto at least one of the end faces (15) of the bundle pack (10).

8. The method as claimed in claim 1, wherein the web of material (20) is imprinted on one side or on both sides during the process.

9. The method as claimed in claim 1, wherein a perforation by way of opening-aid is applied to the web of material (20) or to the portions separated therefrom.

10. The method as claimed in claim 1, wherein a single-layer, unfolded web of material (40) is unwound from a bobbin (41) and folded during the process to form a tubular web of material (20) from which individual sleeves (14) are separated.

11. An apparatus for producing a cuboidal bundle pack (10) for compressible hygiene products (12), wherein a group (11) of hygiene products (12) is capable of being inserted into a sleeve (14), open on two sides, consisting of packaging material, and the sleeve (14) is then capable of being sealed for the purpose of forming a wrapping (13), preferentially closed on all sides, in particular in a method as claimed in one of the preceding claims, comprising:
a transport system for a continuous web of material (20), preferentially paper-based, wound on a bobbin;
one or more stations for applying lines of weakening to the unwound continuous web of material (20);
a separating station for isolating portions for a sleeve (14) from the continuous web of material (20);
a righting station which has been set up to right a portion and to insert a preferentially compressed group (11) of hygiene products (12) into the righted portion via an open side of the sleeve (14); and
a closure station which has been set up to seal opposing end faces (15) of the sleeve (14) by means of an envelope fold in each instance.

12. The apparatus as claimed in claim 11, further comprising a folding station for the folding of folding flaps (16, 17, 18, 19) of the sleeve (14) and a gluing station for applying adhesive onto the folding flaps (16, 17, 18, 19).

13. A bundle pack for a group (11) of compressible hygiene products (12), wherein the group (11) of hygiene products (12) exhibits a cuboidal shape and is surrounded by a sleeve (14) consisting of packaging material, preferentially paper-based, the open sides of which have been at least partially sealed by means of envelope folds, produced by a method comprising the steps of:
unwinding a continuous web of material (20), preferentially paper-based, for the sleeve (14) from a bobbin;
applying lines of weakening to the unwound, continuous web of material (20);
separating a portion for a sleeve (14) from the continuous web of material (20);
righting and inserting the portion and the preferentially compressed group (11) of hygiene products (12) into the righted portion via an open side of the sleeve (14); and
sealing each of opposing end faces (15) of the sleeve (14) by means of an envelope fold.

14. The bundle pack as claimed in claim 13, wherein the sleeve (14) for the bundle pack (10) exhibits an adhesion-bonded seam in the longitudinal direction of a web of material (20) from which the sleeve (14) has been separated.

15. The bundle pack as claimed in claim 13, wherein the sleeve (14) for the bundle pack (10) exhibits lines of weakening, in order to make possible a preferentially sharp-edged fold around the cuboidal group (11) of hygiene products (12).

16. The bundle pack as claimed in claim 13, wherein the end faces (15) of the sleeve (14) with the envelope folds have been sealed in each instance by means of a covering label (23) which has been connected by means of adhesive to the folding flaps (16, 17, 18, 19) folded in the manner of an envelope.

17. The bundle pack as claimed in claim 13, wherein the end faces (15) of the sleeve (14) have been sealed by means of envelope folds overlapping one another, which have been at least partially connected to one another by means of adhesive.

18. The bundle pack as claimed in claim 13, wherein a cold-glue-based adhesive is employed.

19. The bundle pack as claimed in claim 13, wherein the end faces (15) of the sleeve (14) with the envelope folds have been sealed in each instance by means of a self-adhesive label, preferentially paper-based.
